SEQUENCE LISTING (18 sequences)

SEQ ID NO: 1 - protein, 330 aa, Streptococcus pyogenes
NHKVHMHHHHHHADEQEEKAKVRTELIQELAQGLGGIEKKNFPTLGDEDLDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 2 - protein, 292 aa, Streptococcus pyogenes
NHKVHMHHHHHHDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 3 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 4 - protein, 240 aa, Streptococcus pyogenes
GSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 5 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPAGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 6 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPQGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 7 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPAGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 8 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPSGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 9 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPKGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 10 - protein, 102 aa, Streptococcus pyogenes
GERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 11 - protein, 138 aa, Streptococcus pyogenes
GSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPR

SEQ ID NO: 12 - protein, 168 aa, Streptococcus pyogenes
GETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 13 - protein, 192 aa, Streptococcus pyogenes
GLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 14 - protein, 216 aa, Streptococcus pyogenes
GLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 15 - protein, 231 aa, Streptococcus pyogenes
GEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 16 - protein, 72 aa, Streptococcus pyogenes
GSPGLPGPRGEQGPTGPTGPAGPRGLQGLQGLQGERGEQGPTGPAGPRGLQGERGEQGPTGLAGKAGEAGAK

SEQ ID NO: 17 - protein, 48 aa, Streptococcus pyogenes
GSPGLPGPRGEQGPTGPTGPAGPRGLQGLQGLQGERGEQGPTGPAGPR

SEQ ID NO: 18 - protein, 91 aa, Streptococcus pyogenes
MNHKVHMHHHHHHADEQEEKAKVRTELIQELAQGLGGIEKKNFPTLGDEDLDHTYMTKLLTYLQEREQAENSWRKRLLKGIQDHALDLVPR

(19) United States
(12) Patent Application Publication
(10) Pub. No.: US 2024/0343780 A1
(43) Pub. Date: Oct. 17, 2024

(54) POLYNUCLEOTIDE ENCODING A BACTERIAL COLLAGEN-LIKE PROTEIN

(71) Applicant: Evonik Operations GmbH, Essen, DE (US)

(72) Inventors: Andrea Camattari, Newton, MA (US); Christian Banget, Linsengericht, DE (US); Claudia Rollmann, Alzenau, DE (US); Claudia Rinnofner, Hofstaetten/Raab (AT)

(73) Assignee: Evonik Operations GmbH, Essen, DE (US)

(21) Appl. No.: 18/682,348

(22) PCT Filed: Aug. 3, 2022
(86) PCT No.: PCT/EP2022/071834

§ 371 Date: Feb. 8, 2024

(30) Foreign Application Priority Data
Aug. 9, 2021 (EP) 21190325.7

(51) Int. Cl. C07K 14/78; C07K 14/315; C12P 21/02

(52) U.S. Cl. CPC C07K 14/78; C07K 14/3156; C12P 21/02

(57) ABSTRACT

Polynucleotides are made encoding amino acid sequences that are at least 60% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8 or SEQ ID NO:9. The polypeptide is a replicable polypeptide encoding a collagen-like protein and the amino acid sequence includes a deletion of at least 38 amino acids at the N-terminus of the amino acid sequence of SEQ ID NO:1 and respective polypeptides. A fermentative process for secreting bacterial collagen-like proteins in a host is developed.

The present invention relates to secretion of bacterial collagen-like proteins with truncated V-domain, specifically polynucleotides encoding an amino acid sequence that is at least 60%, identical to the amino acid sequence of SEQ ID NO:1, wherein the nucleotide sequence is a replicable nucleotide sequence encoding a collagen-like protein and wherein the amino acid sequence comprises a deletion of at least 38 amino acids at the N-terminus of the amino acid sequence of SEQ ID NO:1 and respective polypeptides as well as a fermentative process for secreting bacterial collagen-like proteins in a host.

Collagen-like proteins (CLPs) of bacterial origin (the most industrially relevant being the product of Streptococcus pyogenes) have considerably interesting mechanical properties, similar to those of higher eukaryotes' collagen proteins, without needing the complex maturing steps required for the eukaryotic counterparts. CLPs present a common structure: two alpha helixes, stabilizing each other, constitute a “V domain”, which is followed by a rod-like, structural collagen domain. After the collagen domain, typically a membrane anchor (GPI-like) is present at the C-terminal end of the protein.

Expression of collagen-like proteins have been attempted in several systems, including Escherichia coli and Saccharomyces cerevisiae. This invention focuses on expression optimization of Streptococcus pyogenes CLP, encoded by sc/2 gene, in the industrial yeast Pichia pastoris.

Although in other hosts (namely Escherichia coli) it has been possible to achieve expression of scl2 (J. Biol. Chem. 277, 27312-27318), and even a reasonably titers once expressed intracellularly (titer approx. at 2 g/L), secreted production in industrially relevant yeast (like P. pastoris) has never been achieved, resulting in a poorly economically viable processes.

For expression in E. coli the construct of choice for such production carries a specific and necessary modification, in order to efficiently remove the potentially immunogenic V domain: such modification consists of a protease cleavage site typically inserted between the V domain and the collagen sequence. Due to this modification, the protein produced by the bacterial host must be extracted from the intracellular fraction and processed with a specific protease to remove the V domain. The mature protein, consisting of only the collagen-like domain, must be purified against the cleaved V domain, the whole intracellular protein content and the protease added to process the immature CLP. Such workflow greatly hinders the cost-effectiveness of the whole process, due to 1) the product of choice must be separated from the whole content of expression host cells, and 2) proteases are typically expensive enzymes.

Therefore, it was an objective of the present invention to provide an improved process for the production of CLP, which is cost-effective and is applicable without the need to add specific proteases for cleavage of the domain.

This invention disclosure provides a solution to achieve a much more cost-effective process, using an industrial workhorse like the yeast Pichia pastoris. Pichia pastoris has been used as a host for other classes of collagen molecules, typically of mammalian origin, as recently reported by Werten and colleagues (Biotechnology Advances 37, Issue 5, 2019, Pages 642-666); however, the of the use of P. pastoris for CLP production has not been described, yet. Moreover, the use of such yeast surprisingly provided a solution to the cleavage of V domain from the mature protein.

In order to understand if the presence of the V domain could be the reason why secreted expression is so inefficient, such domain has been analyzed using a recently published X-ray structure (J. Biol. Chem. 289, 5122-5133), complemented by manual evaluation using a publicly available tool, JPred (http://www.compbio.dundee.ac.uk/jpred). It has been possible to identify the structural determinants of the V domain: this domain has been previously reported to be essential for collagen folding in the vast majority of scientific publications (Protein Science 2010, vol. 19, pp 775-785; J. Biol. Chem., Vol. 280, No. 19, pp. 19343-19349, among many others), although the implication of V domain presence on protein expression has ever being discussed or mentioned. Yu et al. analyzed the role of different fragments of scl2 bacterial subunits (J. Biol. Chem. 286, pp. 18960-18968) in contest of collagen stability, limiting the observation to the structural region but generating also fragments devoid of V domain. Here, no report on improvement of production level is mentioned: this is not surprising, since it is argued that the effect observed as a result of the truncation of V domain is exercised when collagen is secreted, which is an undescribed process for bacterial collagen so far.

Following this hypothesis, a series of truncation were generated: the underlying logic was to maintain a part of V domain, generally reported to be essential for collagen folding, reducing its sequence to minimize any disturbance to the production machinery within the cell, and so being able to secrete significant amount of protein in the supernatant. Upon cloning and introduction of these constructs in P. pastoris, surprisingly it was realized that secretion of CLPs was greatly improved when the V domain was truncated from the original complete sequence. Partial removal of the V domain allowed to significantly increase protein production in P. pastoris; surprisingly, complete removal of the V domain was not as efficient as partial truncation of such domain.

The result of this invention can be technically applied to any modified sequence of scl2 in the collagen domain, as it is intended as a facilitated sequence to promote either efficient translation or efficient transiting through the secretion machinery in Pichia pastoris.

This invention describes a novel process to produce bacterial collagen-like proteins (CLPs) in the methylotrophic yeast Pichia pastoris. The key features of such process, compared to the current process known from the prior art: 1) proteins are secreted in culture supernatant, allowing to reach a high titer (>5 g/L), in a low-cost medium; 2) proteins are easily purified from the supernatant, since no complex component is present in the cultivation medium.

Surprisingly, the purified product from supernatants of Pichia pastoris cultivation secreting Scl2p, showed an unexpected profile, compatible with mature collagen-like sequences. Further analysis showed how intracellular enzymes, most likely the processing protease Kex2p, are capable to remove the V domain protein sequence without any need of an additional protease step. In addition, in order to modify a cleavage site present in the final product, resulting in significant accumulation of degradation products, the protein sequence has been mutated to engineer such cleavage site and abolish degradation. Unexpectedly, the most efficient performance was obtained when an apolar amino acid (valine, in the wild-type sequence) was mutated to a polar amino acid (glutamine). The described process, therefore:
1) Is more competitive than the state-of-art production process, allowing to accumulate the product outside the cell, therefore allowing to avoid any cell disruption to isolate the protein of interest;
2) Surprisingly, no additional step of digestion with proteases is required to remove the undesired V domain for the main product. Such technical improvement allows to further improve process cost-effectiveness, allowing to obtain the desired product directly in cell culture supernatant.
3) The described process applies (also) to constructs carrying truncation of V domain.
4) The described process describes also typical fermentation byproducts, as well protein engineering to minimize the most abundant degradation products

Therefore, the invention provides a novel fermentative process for secreting a bacterial collagen-like protein and respective nucleotide sequences and polypeptides.

The invention relates to a polynucleotide encoding an amino acid sequence that is at least 60%, identical to the amino acid sequence of SEQ ID NO:1, wherein the polynucleotide is a replicable a polynucleotide encoding a collagen-like protein and wherein the amino acid sequence comprises a deletion of at least 38 amino acids at the N-terminus of the amino acid sequence of SEQ ID NO:1.

It was a surprising finding that truncated variants of the collagen-like protein, including variants with a truncated V-domain or without any V-domain lead to increased production of collagen-like protein and secretion into the fermentation medium.

It is preferred, when the amino acid sequence comprises a deletion of between 38 and 90 amino acids at the N-terminus of the amino acid sequence of SEQ ID NO:1. This includes a complete deletion of the N-terminal V-domain (comprising 90 amino acids) and different truncations of the V-domain of at least 38 amino acids. In a preferred embodiment, the amino acid sequence comprises a deletion of between 38 and 74 amino acids or between 38 and 89 amino acids at the N-terminus of the amino acid sequence of SEQ ID NO:1.

In a preferred embodiment, the amino acid sequence that is at least 60%, identical to the amino acid sequence of SEQ ID NO:2, SEQ ID NO:3, SEQ ID NO:4, SEQ ID NO:5, SEQ ID NO:6, SEQ ID NO:7, SEQ ID NO:8 or SEQ ID NO:9.

In a further configuration, the amino acid sequence that is at least 65%, or 70%, or 75%, or 80%, or 85% identical to the amino acid sequence of SEQ ID NO:2, SEQ ID NO:3, SEQ ID NO:4, SEQ ID NO:5, SEQ ID NO:6, SEQ ID NO:7, SEQ ID NO:8 or SEQ ID NO:9.

In a preferred configuration, the polynucleotide encodes an amino acid sequence that is at least 90%, 92%, 94%, 96%, 97%, 98%, 99% or 100%, preferably 97%, particularly preferably 98%, very particularly preferably 99%, and extremely preferably 100%, identical to the amino acid sequence of SEQ ID NO:2, SEQ ID NO:3, SEQ ID NO:4, SEQ ID NO:5, SEQ ID NO:6, SEQ ID NO:7, SEQ ID NO:8 or SEQ ID NO:9.

In a preferred embodiment of the present invention the polynucleotide is a replicable nucleotide sequence encoding the collagen-like protein from Streptococcus pyogenes.

The invention correspondingly also relates to a polynucleotide and nucleic acid molecules comprising such sequences and encoding polypeptide variants of SEQ ID NO:2 to 9, which contain one or more insertion(s) or deletion(s). Preferably, the polypeptide contains a maximum of 5, a maximum of 4, a maximum of 3, or a maximum of 2, insertions or deletions of amino acids.

The invention further relates to a polypeptide comprising an amino acid sequence encoded by the nucleotide sequence according to the invention.

The invention also relates to a mixture of polypeptides comprising one of the polypeptide variants of SEQ ID NO:2 to 9 and on or more of the truncated variants of the collagen-like protein of SEQ ID NO:10 to 17. Those related to specific byproducts from the fermentation.

In another specific embodiment, the polypeptide contains at least one amino acid exchange at position 132 or 135.

The invention further relates to plasmids and vectors that comprise the nucleotide sequences according to the invention and optionally replicate in microorganisms of the genera Pichia, Corynebacterium, Pseudomonas or Escherichia or are suitable therefor. In a preferred configuration, the vector comprising the nucleotide sequences according to the present invention is suitable for replication in yeast of the genus Pichia pastoris.

The invention further relates to microorganisms of the genera Pichia, Corynebacterium, Pseudomonas or Escherichia that comprise the polynucleotides, vectors and polypeptides according to the invention. Preferred microorganisms are Pichia pastoris, Brevibacillus choshinensis or Corynebacterium glutamicum.

The invention further relates to a microorganism according to the invention, characterized in that the polypeptide according to the invention is integrated in a chromosome. Homologous recombination permits, with use of the vectors according to the invention, the exchange of DNA sections on the chromosome for polynucleotides according to the invention which are transported into the cell by the vector. For efficient recombination between the ring-type DNA molecule of the vector and the target DNA on the chromosome, the DNA region that is to be exchanged containing the polynucleotide according to the invention is provided at the ends with nucleotide sequences homologous to the target site; these determine the site of integration of the vector and of exchange of the DNA.

The present invention provides a microorganism of the species P. pastoris, E. coli, P. putida or C. glutamicum comprising any of the nucleotide sequences as claimed or any of the polypeptides as claimed or any of the vectors as claimed.

The microorganism may be a microorganism in which the nucleotide sequence is present in overexpressed form.

The microorganism may be characterized in that the microorganism has the capability of producing and secreting a fine chemical. The fine chemical being preferably a bacterial collagen-like protein.

Overexpression is taken to mean, generally, an increase in the intracellular concentration or activity of a ribonucleic acid, a protein (polypeptide) or an enzyme, compared with the starting strain (parent strain) or wild-type strain, if this is the starting strain. A starting strain (parent strain) is taken to mean the strain on which the measure leading to the overexpression was carried out.

In the overexpression, the methods of recombinant overexpression are preferred. These include all methods in which a microorganism is produced using a DNA molecule provided in vitro. Such DNA molecules comprise, for example, promoters, expression cassettes, genes, alleles, encoding regions etc. These are converted into the desired microorganism by methods of transformation, conjugation, transduction or like methods.

The extent of the expression or overexpression can be established by measuring the amount of the mRNA transcribed by the gene, by determining the amount of the polypeptide, and by determining the enzyme activity.

Disclosed is a fermentative process for secreting a bacterial collagen-like protein in a host comprising the following steps:
a) fermentation of a microorganism according to the present invention in a medium,
b) accumulation of the bacterial collagen-like protein in the medium, wherein a fermentation broth is obtained.

The use of such a process according to the invention leads, as shown in the Examples, to an extraordinary increase in product concentration and secretion of bacterial collagen-like protein compared with the respective starting strain.

The culture medium or fermentation medium that is to be used must appropriately satisfy the demands of the respective strains. Descriptions of culture media of various microorganisms are contained in the handbook “Manual of Methods for General Bacteriology” of the American Society for Bacteriology (Washington D.C., USA, 1981). The terms culture medium and fermentation medium or medium are mutually exchangeable.

As carbon source, sugars and carbohydrates can be used, such as, e.g., glucose, sucrose, lactose, fructose, maltose, molasses, sucrose-containing solutions from beet sugar or sugar cane processing, starch, starch hydrolysate and cellulose, oils and fats, such as, for example, soybean oil, sunflower oil, groundnut oil and coconut fat, fatty acids, such as, for example, palmitic acid, stearic acid and linoleic acid, alcohols such as, for example, glycerol, methanol and ethanol, and organic acids, such as, for example, acetic acid or lactic acid.

As nitrogen source, organic nitrogen compounds such as peptones, yeast extract, meat extract, malt extract, corn-steep liquor, soybean meal and urea or inorganic compounds such as ammonium sulphate, ammonium chloride, ammonium phosphate, ammonium carbonate and ammonium nitrate can be used. The nitrogen sources can be used individually or as a mixture.

As phosphorus source, phosphoric acid, potassium dihydrogenphosphate or dipotassium hydrogenphosphate or the corresponding sodium-containing salts can be used.

The culture medium must, in addition, contain salts, for example in the form of chlorides or sulphates of metals such as, for example, sodium, potassium, magnesium, calcium and iron, such as, for example, magnesium sulphate or iron sulphate, which are necessary for growth. Finally, essential growth substances such as amino acids, for example homoserine and vitamins, for example thiamine, biotin or pantothenic acid, can be used in addition to the above-mentioned substances.

Said starting materials can be added to the culture in the form of a single batch or supplied in a suitable manner during the culturing.

Basic compounds such as sodium hydroxide, potassium hydroxide, ammonia or ammonia water, or acid compounds such as phosphoric acid or sulphuric acid, are used in a suitable manner for pH control of the culture. The pH is generally adjusted to 6.0 to 8.5, preferably 6.5 to 8. For control of foam development, antifoams can be used, such as, for example, polyglycol esters of fatty acids. For maintaining the stability of plasmids, suitable selectively acting substances such as, for example, antibiotics, can be added to the medium. The fermentation is preferably carried out under aerobic conditions. In order to maintain said aerobic conditions, oxygen or oxygen-containing gas mixtures such as, for example, air, are introduced into the culture. The use of liquids that are enriched with hydrogen peroxide is likewise possible. Optionally, the fermentation is carried out at superatmospheric pressure, for example at a superatmospheric pressure of 0.03 to 0.2 MPa. The temperature of the culture is usually 20° C. to 45° C., and preferably 25° C. to 40° C., particularly preferably 30° C. to 37° C. In the case of batch or fed-batch processes, the culturing is preferably continued until an amount sufficient for the measure of obtaining the desired organic chemical compound has formed. This goal is usually reached within 10 hours to 160 hours. In continuous processes, longer culture times are possible. Owing to the activity of the microorganisms, enrichment (accumulation) of the fine chemicals in the fermentation medium and/or in the cells of the microorganisms occurs.

Examples of suitable fermentation media may be found, inter alia, in patent documents U.S. Pat. Nos. 5,770,409, 5,990,350, 5,275,940, WO 2007/012078, U.S. Pat. No. 5,827,698, WO 2009/043803, U.S. Pat. No. 5,756,345 or U.S. Pat. No. 7,138,266; appropriate modifications may optionally be carried out to the requirements of the strains used.

The process may be characterized in that it is a process which is selected from the group consisting of batch process, fed-batch process, repetitive fed-batch process and continuous process.

The performance of the processes or fermentation processes according to the invention with respect to one or more of the parameters selected from the group of concentration (compound formed per volume), yield (compound formed per carbon source consumed), volumetric productivity (compound formed per volume and time) and biomass-specific productivity (compound formed per cell dry mass or bio dry mass and time or compound formed per cell protein and time) or other process parameters and combinations thereof, is increased by at least 0.5%, at least 1%, at least 1.5% or at least 2%, based on processes or fermentation processes with microorganisms in which the promoter variant according to the invention is present.

A fermentation broth is taken to mean, in a preferred embodiment, a fermentation medium or nutrient medium in which a microorganism was cultured for a certain time and at a certain temperature. The fermentation medium, or the media used during the fermentation, contains/contain all substances or components that ensure production of the desired compound and typically ensure growth and/or viability.

On completion of the fermentation, the resultant fermentation broth accordingly contains
a) the biomass (cell mass) of the microorganism resulting from growth of the cells of the microorganism,
b) the desired fine chemical formed in the course of the fermentation,
c) the organic by-products possibly formed in the course of the fermentation, and
d) the components of the fermentation medium used, or of the starting materials, that are not consumed by the fermentation, such as, for example, vitamins such as biotin, or salts such as magnesium sulphate.

The organic by-products include substances which are generated in addition to the respective desired compound by the microorganisms used in the fermentation and are possibly secreted.

The fermentation broth is withdrawn from the culture vessel or the fermentation container, optionally collected, and used for providing a product in liquid or solid form containing the fine chemical. The expression “obtaining the fine chemical-containing product” is also used therefor. In the simplest case, the fine chemical-containing fermentation broth withdrawn from the fermentation container is itself the product obtained.

By way of one or more of the measures selected from the group
a) partial (>0% to <80%) to complete (100%) or virtually complete (≥80%, ≥90%, ≥95%, ≥96%, ≥97%, ≥98%, ≥99%) removal of the water,
b) partial (>0% to <80%) to complete (100%) or virtually complete (≥80%, ≥90%, ≥95%, ≥96%, ≥97%, ≥98%, ≥99%) removal of the biomass, wherein this is optionally inactivated before the removal,
c) partial (>0% to <80%) to complete (100%) or virtually complete (≥80%, ≥90%, ≥95%, ≥96%, ≥97%, ≥98%, ≥99%, ≥99.3%, ≥99.7%) removal of the organic by-products formed in the course of the fermentation, and
d) partial (>0%) to complete (100%) or virtually complete (≥80%, ≥90%, ≥95%, ≥96%, ≥97%, ≥98%, ≥99%, ≥99.3%, ≥99.7%) removal of the components of the fermentation medium used or the starting materials that are not consumed by the fermentation,
a concentration or purification of the desired organic chemical compound is achieved from the fermentation broth. In this manner, products are isolated that have a desired content of the compound.

The partial (>0% to <80%) to complete (100%) or virtually complete (≥80% to <100%) removal of the water (measure a)) is also termed drying.

In a variant of the process, by complete or virtually complete removal of the water, the biomass, the organic by-products and the non-consumed components of the fermentation medium used, pure (≥80% by weight, ≥90% by weight) or high-purity (≥95% by weight, ≥97% by weight, ≥99% by weight) product forms of the desired organic chemical compound, preferably bacterial collagen-like protein, are successfully arrived at. For the measures according to a), b), c) or d), a great variety of technical instructions are available in the prior art.

In the case of processes for producing bacterial collagen-like protein processes are preferred in which products are obtained that do not contain any components of the fermentation broth. These products are used, in particular, in human medicine, in the pharmaceuticals industry, and in the food industry.

The process according to the invention serves for the fermentative production and secretion of bacterial collagen-like protein.

The invention finally relates to use of the microorganism according to the invention for the fermentative production and secretion of bacterial collagen-like protein.

### EXAMPLES

A) Fermentation in Pichia pastoris

To achieve the goal of producing a protein, which may easily be purified from the supernatant, the sequence of gene scl2 from Streptococcus pyogenes, encoding for a collagen-like protein, has been codon optimized using different algorithms, and cloned in a secretion vector pBSY5S1Z (Bisy GmbH, Austria) for Pichia pastoris; such vector triggers protein expression in dependence of low level of glycerol as carbon source in the medium. Upon transformation in Pichia pastoris following standard protocol (Cereghino et al., Biotechniques (2005), 38(1): p44) and application of expression protocol in fed-batch mode, protein corresponding to Scl2p was detected in the supernatant of cell culture. Scl2p constructs according to SEQ ID NO:1 to SEQ ID NO:4 were used.

Since V domain has been reported to be potentially interacting with human receptor or ligands (Squeglia et al., Journal of Biological Chemistry (2014), 289:p 5122), the construct for expression in Pichia pastoris carries a protease cleavage site between the V domain and the mature collagen-like domain. Such domain must be removed digesting the product with a protease like trypsin.

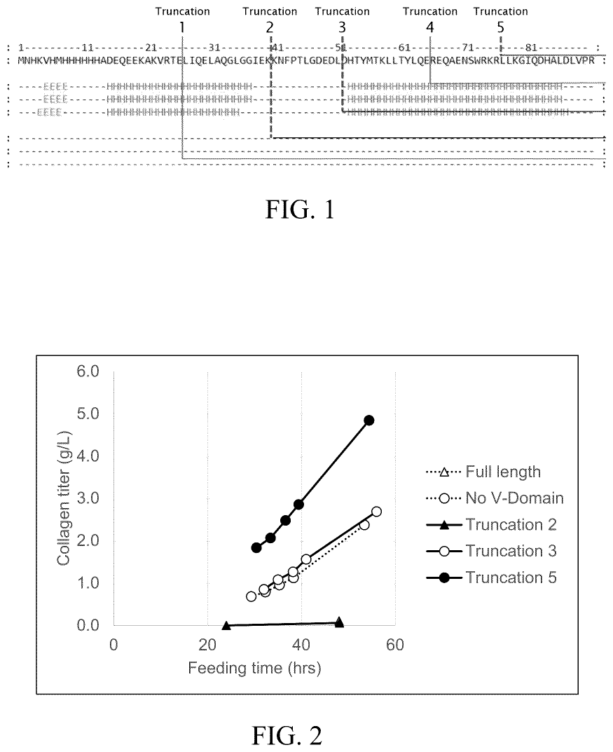

As mentioned, it was hypothesized that the presence of V-domain might represent a hindrance to efficient protein expression; hence, several truncated versions of V-domain were generated, following the structural architecture of V-domain itself. FIG. 1 reports the structural description of the different truncations, with reference to the alpha helix structures of V domain (indicated by H in the picture).

Different strains of Pichia pastoris, carrying the different construct, have been cultivated over-weekend in 100 mL shake flask containing 10 mL of BMGY media (2% Peptone, 1% Yeast Extract, 100 mM Potassium Phosphate pH 6.0, 1.34% Yeast Nitrogen Base (w/o AA), 0.4 μg/mL Biotin, 1% Glycerol). Subsequently, 2% of the culture have been transferred in a new 500 mL shake flask containing 50 mL of BMGY for an overnight cultivation. 4.5% of such culture have been used to inoculate a 2 L steel fermenter, containing 1 liter of production medium (table 1). Fermentation was run at 28° C., pH 5.5, pressure 800 mbar, controlling dissolved oxygen at 20%; once the batch phase ended (approximately 20 hrs), a fed-batch phase was performed feeding a solution of 80% glycerol at 2.1 g/h, ramping to 5.7 g/h in 20 hours; then, feeding rate was ramped from 5.7 g/h to 12 g/h in 15 hrs. Feeding rate was then kept constant for the remaining fed-batch phase (total duration of fed batch approximately 50 hrs).

TABLE 1
Production medium

| Ingredient | Amount [g/kg] |
|---|---|
| Glycerol | 20.0 |
| tab-H₂O | 950.0 |
| CaSO₄ * 2H₂O | 0.17 |
| NaCl | 0.22 |
| KH₂PO₄ | 22.00 |
| K₂SO₄ | 2.86 |
| MgSO₄ * 7H₂O | 14.0 |
| PTM1a | 2.18 ml |
| PTM1b | 2.18 ml |
| Biotin (100%) | 0.0009 |
| Phosphoric Acid, 85% | 5.4 |
| Antischaum (Delamex) | 0.2 |

TABLE 2
PTM1A formulation

| | |
|---|---|
| H₃BO₃ | 0.040 g/L |
| CoCl₂ 6H₂O | 1.840 g/L |
| NaJ | 2.130 g/L |
| Na₂MoO₄ 2H₂O | 0.400 g/L |

TABLE 3
PTM1B formulation

| | | |
|---|---|---|
| CuSO₄ 5H₂O | 11.980 | g/L |
| FeSO₄ 7H₂O | 130.000 | g/L |
| MnSO₄ H₂O | 6.000 | g/L |
| ZnSO₄ 7H₂O | 84.360 | g/L |
| H₂SO₄, 96% | 11.500 | g/L |

Fermentation of a Pichia pastoris strain expressing a representative clone for the most promising V-domain variant has been performed as described above. Upon fermentation, supernatant has been separated from biomass and further analyzed via RP-HPLC to identify cleavage products. The sample was incubated for 17 h at 15° C. and 1000 rpm in a Thermomixer with 0.4 g/L of rTrypsin and analyzed using a RP-HPLC method to determine protein length (Agilent Zorbax 300 SB-C8 4,6×150 mm, 3.5 μm particle size).

Supernatant samples from at least 5 independent clones carrying the different truncated constructs were loaded on SDS-PAGE, clearly showing the general trend of improving expression level from truncation 1 to truncation 5.

Selected clones, carrying the truncation versions evaluated to be the most promising, have been cultivated in bioreactor (see fermentation protocol above). FIG. 2 clearly shows a definite improvement in protein production comparing a “No V domain” version versus truncation 3 and 5. Titers obtained for full length V domain constructs were lower or similar as for the “No V domain” versions. The final collagen concentration (g/l) is summarized in table 4.

TABLE 4
final collagen concentration after fermentation in Pichia pastoris

| Collagen version | Final collagen concentration (g/L) | CLP sequence |
|---|---|---|
| Full length | 0.1 | SEQ ID NO: 1 |
| No V-domain | 2.37 | SEQ ID NO: 4 |
| Truncation 2 | 0.06 | |
| Truncation 3 | 2.69 | SEQ ID NO: 2 |
| Truncation 5 | 4.85 | SEQ ID NO: 3 |

### HPLC Analysis

Fermentation of a Pichia pastoris strain containing a V-domain truncation has been performed as described above. Upon fermentation, supernatant has been separated from biomass via centrifugation (12000 g, 5 mins at room temperature); supernatants from truncation 3 or truncation 5 of V-domain were analyzed before and after trypsin digest, to compare the result to a CL standard solution (no V-domain).

In particular, samples were incubated for 17 h at 15° C. and 1000 rpm in a Thermomixer with varying concentrations of rTrypsin (to avoid over digest) and analyzed using a RP-HPLC method to determine protein length (Agilent Zorbax 300 SB-C8 4,6×150 mm, 3.5 μm particle size).

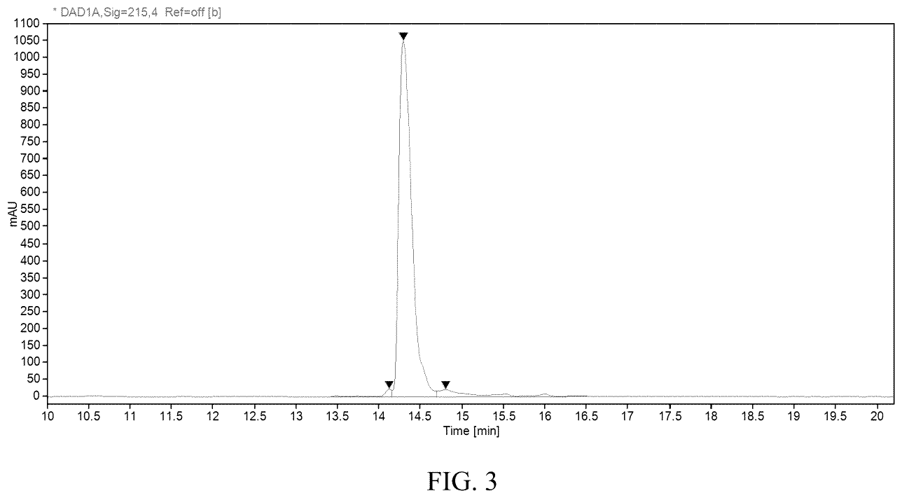

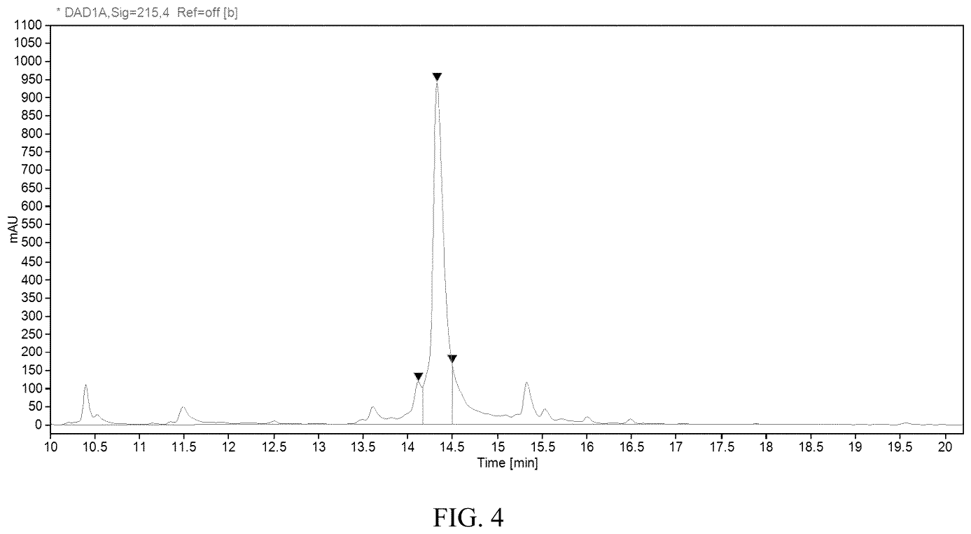

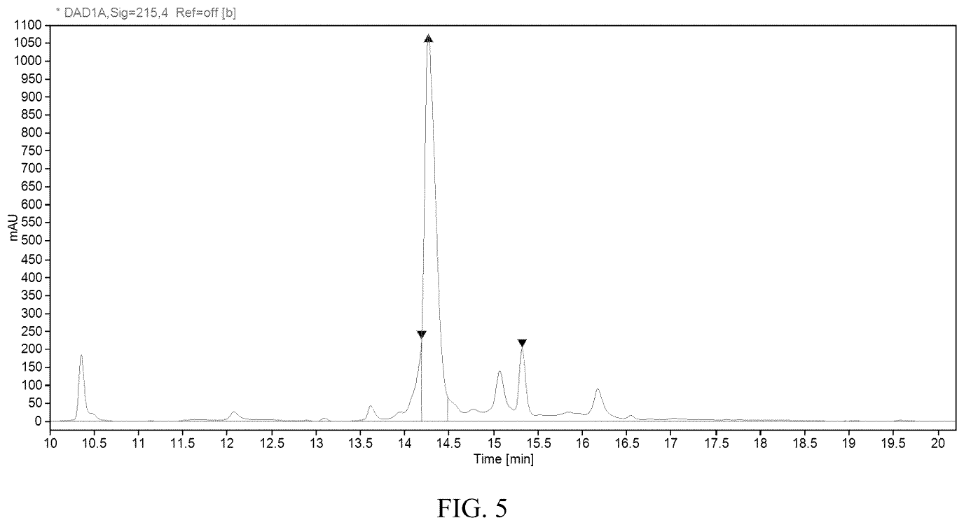

As shown in FIGS. 3-5, no difference on protein length were detected comparing bacterial collagen derived from a truncation 5 construct (FIG. 4) and a bacterial collagen standard (FIG. 3), which does not carry any V-domain. No difference was also observed when the fermentation sample was incubated with 0.4 g/L of trypsin (FIG. 5), indicating that the V-domain was already processed.

The retention times (RT) of the chromatograms of FIGS. 3-5 are summarized in tables 5-7, where table 5 shows RT for the purified product after trypsin digestion, table 6 for truncation 5 before trypsin digestion and table 7 for truncation 5 after trypsin digestion.

TABLE 5
purified product after trypsin digestion

| | | | | Width | Area |
|---|---|---|---|---|---|
| RT [min] | comment | Area | Height | [min] | % |
| 14.18 | | 3248.4 | 224.5 | 0.24 | 17.7 |
| 14.26 | Target collagen product | 9581.7 | 1074.6 | 0.14 | 52.1 |
| 15.31 | | 5564.1 | 201.8 | 0.34 | 30.2 |

TABLE 6
Truncation 5, before trypsin digestion

| | | | | Width | Area |
|---|---|---|---|---|---|
| RT [min] | comment | Area | Height | [min] | % |
| 14.11 | | 3129.7 | 116.0 | 0.33 | 20.6 |
| 14.32 | Target collagen product | 8457.9 | 938.9 | 0.13 | 55.7 |
| 15.49 | | 3593.2 | 163.8 | 0.37 | 23.7 |

TABLE 7
Truncation 5, after trypsin digestion

| | | | | Width | Area |
|---|---|---|---|---|---|
| RT [min] | comment | Area | Height | [min] | % |
| 14.18 | | 3248.4 | 224.5 | 0.24 | 17.7 |
| 14.26 | Target collagen product | 9581.7 | 1074.6 | 0.14 | 52.1 |
| 15.31 | | 5564.1 | 201.6 | 0.34 | 30.2 |

Analyzing fermentation supernatants via HPLC, besides a large peak corresponding to the target product, a typical byproduct profile was detected (see table 8). Further analysis with LC/MS allowed to identify the nature of the peaks as well as all molecular masses involved, allowing to identify the sequence of every product generated. The sequences of the Byproducts are summarized in SEQ ID NO:10 to SEQ ID NO:17.

TABLE 8
Byproducts after fermentation in supernatant

| | Size (kDa) | Amino acids | RT (mins) | Amount |
|---|---|---|---|---|
| Full length | 22840 | 240 | 13.73-13.81 | |
| Byproduct 1 | 9916 | 102 | 9.91-9.94 | >5% (main peak) |
| Byproduct 2 | 12941 | 138 | 14.64-14.70 | >5% (main peak) |
| Byproduct 3 | 16096 | 168 | 10.69 | >1% |
| Byproduct 4 | 18317 | 192 | 11.08-11.13 | >5% (main peak) |
| Byproduct 5 | 20660 | 216 | 12.89-12.93 | >1% |
| Byproduct 6 | 22020 | 231 | 13.50-13.70 | >1% |
| Byproduct 7 | 6761 | 72 | 15.39 | >1% |
| Byproduct 8 | 4539 | 48 | 15.78-15.81 | >5% (main peak) |

Focusing on the most relevant degradation products in terms of abundance compared to the expected product, accurate mass identification allowed to hypothesize the cleavage site along the protein sequence. As such, an engineering strategy to substitute the key amino acids to prevent cleavage was tested. In particular, it was hypothesized that the sequence VGPR (Val-Gly-Pro-Arg, with valine at position 132) could be corrected in the position −4 (=V, Valine) or −1 (=R, Arginine): several mutant sequences were therefore generated and tested (see tables 9 and 10). Previously, a somehow similar cleavage was reported for gelatin, where the sequence MGPR (Met-Gly-Pro-Arg) was recognized to yield degradation, and was corrected to RGPM (Arg-Gly-Pro-Met), which generally maintain the amino acid polarity (Werten et al., Yeast 15 (1999), p 1087-1096).

TABLE 9
different mutants of cleavage site

| Sequence | Description |
|---|---|
| VGPR | Wild type sequence |
| AGPR | Proposed mutant |
| QGPR | Proposed mutant |
| VGPA | Proposed mutant |
| SGPR | Proposed mutant |
| VGPK | Proposed mutant |

Even though in all cases the intramolecular cleavage product were significantly reduced via either SDS-PAGE or HPLC, quite surprisingly substituting an apolar amino acid (Valine at position 132) with a polar amino acid (glutamine, in mutant QGPR, table 9) led to the best performance in terms of cleavage site correction and product titer. The abovementioned mutations have been introduced via site-directed mutagenesis of the wild-type sequence, using the expression construct as template. Upon verification of successful mutagenesis, the modified expression vector has been introduced in Pichia pastoris similarly as described above. All constructs (according to SEQ ID NO: 5-SEQ ID NO:10) have been cultivated in bioreactor as described below. Analysis via SDS-PAGE (small scale cultivation) and RP-HPLC demonstrated how the degradation products, previously derived by proteolysis at the hypothesized cleavage site, were completely absent upon modification of such cleavage site. SDS-PAGE analysis from 4 independent cultivations of the most promising mutants showed different cleavage patterns than the wild-type sequence (AGPR, QGPR and VGPA). Additionally, a mutant deleted in the YPS1 protease locus (Δyps1 mutant) was analyzed. In all cases, clones carrying modified versions of the wild-type sequence do not present degradation band, detectable in supernatants from cultivation with clones carrying the wild-type sequence. The results from the SDS-PAGE are summarized in table 10. The results were confirmed via RP-HPLC analysis.

TABLE 10
SDS-PAGE analysis from 4 independent cultivations
of the most promising mutants

| Mutant | Presence of degradation bands |
|---|---|
| Wild type sequence | Yes (<10 kDa) |
| AGPR mutant | No |
| QGPR mutant | No |
| VGPA mutant | No |
| Δyps1 mutant | Yes (<10 kDa) |

B) Fermentation in Brevibacillus choshinensis

The full-length collagen-like protein, a truncated variant (truncation 3) and the no-V-domain variant (based on the gene sc/2 from Streptococcus pyogenes as used in Example A) were also expressed in Brevibacillus choshinensis. Therefore, the corresponding DNA sequences were cloned into a suitable secretion vector for B. choshinensis. Transformation of B. choshinensis with the new constructed plasmids was done according to Mizukami et al. 2010 (Curr Pharm Biotechnol 2010, 13:151-258).

The B. choshinensis strains were analyzed for their ability to produce the different collagen proteins in batch cultivations at 33° C. and pH 7 using the DASGIP@ parallel bioreactor system from Eppendorf (Hamburg, Germany). The fermentation was performed using 1 L reactors. The production medium (TM medium, Biomed Res Int 2017, 2017: 5479762) contained 10 g/L glucose. Upon fermentation, supernatant has been separated from biomass by centrifugation and was used for SDS PAGE analysis. For all three variants, collagen-like protein was produced.

C) Fermentation in Corynebacterium glutamicum

The full-length collagen-like protein and the no-V-domain variant (based on the gene sc/2 from Streptococcus pyogenes as used in Example A) were also expressed in Corynebacterium glutamicum. Therefore, the corresponding DNA sequences were cloned together with an upstream located signal peptide for protein secretion into a shuttle vector for C. glutamicum (Biotechnology Techniques 1999, 13: 437-441.). The C. glutamicum strain ATCC 13032 was transformed with the new constructed plasmids by means of electroporation as described by Ruan et al. (Biotechnology Letters 2015, 37: 2445-2452).

The C. glutamicum strains were analyzed for their ability to produce the different collagen proteins in fed-batch cultivations at 30° C. and pH 7 using the DASGIP® parallel bioreactor system from Eppendorf (Hamburg, Germany). The fermentation was performed using 1 L reactors. The production medium contained 20 g/L glucose in the batch phase and the fed-batch phase was run with a glucose feed of 4 g/L*h. Upon fermentation, supernatant has been separated from biomass by centrifugation and was used for HPLC analysis. For both variants, collagen protein was produced. For the truncated variant of the collagen-like protein, product titer was higher as for the full-length variant.

Protein sequences

| | |
|---|---|
| SEQ ID NO: 1 | Streptococcus pyogenes Collagen-like protein (CLP), full length protein |
| SEQ ID NO: 2 | Streptococcus pyogenes CLP, truncation 3 |
| SEQ ID NO: 3 | Streptococcus pyogenes CLP, truncation 5 |
| SEQ ID NO: 4 | Streptococcus pyogenes CLP, no V-domain |
| SEQ ID NO: 5 | Streptococcus pyogenes CLP, truncation 5 (AGPR mutant) |
| SEQ ID NO: 6 | Streptococcus pyogenes CLP, truncation 5 (QGPR mutant) |
| SEQ ID NO: 7 | Streptococcus pyogenes CLP, truncation 5 (VGPA mutant) |
| SEQ ID NO: 8 | Streptococcus pyogenes CLP, truncation 5 (SGPR mutant) |
| SEQ ID NO: 9 | Streptococcus pyogenes CLP, truncation 5 (VGPK mutant) |
| SEQ ID NO: 10 | Streptococcus pyogenes CLP, byproduct 1 |
| SEQ ID NO: 11 | Streptococcus pyogenes CLP, byproduct 2 |
| SEQ ID NO: 12 | Streptococcus pyogenes CLP, byproduct 3 |
| SEQ ID NO: 13 | Streptococcus pyogenes CLP, byproduct 4 |
| SEQ ID NO: 14 | Streptococcus pyogenes CLP, byproduct 5 |
| SEQ ID NO: 15 | Streptococcus pyogenes CLP, byproduct 6 |
| SEQ ID NO: 16 | Streptococcus pyogenes CLP, byproduct 7 |
| SEQ ID NO: 17 | Streptococcus pyogenes CLP, byproduct 8 |
| SEQ ID NO: 18 | Streptococcus pyogenes CLP, fragment from figure 1 |

1. A polynucleotide encoding an amino acid sequence that is at least 60% identical to the amino acid sequence of SEQ ID NO: 1, wherein the polynucleotide is a replicable polynucleotide encoding a collagen-like protein, and wherein the amino acid sequence comprises a deletion of at least 38 amino acids at the N-terminus of the amino acid sequence of SEQ ID NO: 1.

2. The polynucleotide according to claim 1, wherein the amino acid sequence comprises a deletion of between 38 and 90 amino acids at the N-terminus of the amino acid sequence of SEQ ID NO:1.

3. The polynucleotide according to claim 1, encoding an amino acid sequence that is at least 60% identical to the amino acid sequence of SEQ ID NO:2, SEQ ID NO:3, SEQ ID NO:4, SEQ ID NO:5, SEQ ID NO:6, SEQ ID NO:7, SEQ ID NO:8 or SEQ ID NO:9.

4. The polynucleotide according to claim 1, encoding an amino acid sequence that is at least 90% identical to the amino acid sequence of SEQ ID NO:2, SEQ ID NO:3, SEQ ID NO:4, SEQ ID NO:5, SEQ ID NO:6, SEQ ID NO:7, SEQ ID NO:8 or SEQ ID NO:9.

5. The polynucleotide according to claim 1, wherein a nucleotide sequence is a replicable nucleotide sequence encoding the collagen-like protein from Streptococcus pyogenes.

6. A vector, comprising:
the polynucleotide according to claim 1.

7. A polypeptide, comprising:
an amino acid sequence encoded by the polynucleotide according to claim 1.

8. A microorganism, comprising:
the polynucleotide according to claim 1.

9. The microorganism according to claim 8, wherein the microorganism is of the genus Pichia, Brevibacillus, Bacillus, Escherichia or Corynebacterium.

10. The microorganism according to claim 9, wherein the polynucleotide is present in overexpressed form.

11. The microorganism according to claim 8, wherein the microorganism has the capability of secreting a bacterial collagen-like protein.

12. A fermentative process for secreting a bacterial collagen-like protein in a host, comprising:
a) fermentation of the microorganism according to claim 8 in a medium,
b) accumulation of the bacterial collagen-like protein in the medium, wherein a fermentation broth is obtained.

13. The process according to claim 12, wherein it is a process which is selected from the group consisting of batch process, fed-batch process, repetitive fed-batch process and continuous process.

14. (canceled)

15. The polynucleotide according to claim 1, encoding an amino acid sequence that is 100% identical to the amino acid sequence of SEQ ID NO:2, SEQ ID NO:3, SEQ ID NO:4, SEQ ID NO:5, SEQ ID NO:6, SEQ ID NO:7, SEQ ID NO:8 or SEQ ID NO:9.

16. The microorganism according to claim 8, wherein the microorganism is Pichia pastoris, Brevibacillus choshinensis or Corynebacterium glutamicum.
